(19) United States
(12) Patent Application Publication
(10) Pub. No.: US 2007/0243109 A1
(43) Pub. Date: Oct. 18, 2007

(54) MICRO-SCALE HEATING MODULE

(75) Inventors: Jyh-Jian Chen, Taoyuan County (TW); Jhy-Wen Wu, Hsinchu City (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, omitted

(73) Assignee: INDUSTRIAL TECHNOLOGY RESEARCH INSTITUTE, Hsinchu (TW)

(21) Appl. No.: 11/467,929

(22) Filed: Aug. 29, 2006

(30) Foreign Application Priority Data
Apr. 14, 2006 (TW) 95113331

(51) Int. Cl. B01L 3/00; C12M 1/34

(52) U.S. Cl. 422/99; 435/288.5; 435/303.1; 422/102

(57) ABSTRACT

A micro-scale heating module for heating a micro-fluidic chip is provided. The micro-fluidic chip typically includes an inlet, an outlet and a working region between the inlet and the outlet. The micro-scale heating module includes a preheating part and a heating part. The preheating part is correspondingly disposed on the inlet of the micro-fluidic chip. The heating part connects with the preheating part and surrounds the working region of the micro-fluidic chip in order to make the temperature distribution in the working region uniform. The advantages of the micro-scale heating module include simplicity in design, large flow rate in the working region and large working surface. Therefore, the micro-scale heating module can be used in cell culture, cell-to-pharmaceutical test, biochemical test and so on.

### CROSS-REFERENCE TO RELATED APPLICATION

This application claims the priority benefit of Taiwan application serial no. 95113331, filed Apr. 14, 2006. All disclosure of the Taiwan application is incorporated herein by reference.

### BACKGROUND OF THE INVENTION

1. Field of the Invention

The present invention relates to a heating module, and more particularly, to a micro-scale heating module.

2. Description of Related Art

Micro-fluidic techniques have found a variety of applications in devices used for conventional biochemical analysis including, for example, micro-pumps, micro-valves, micro-filters, micro-mixers, micro-tubes and micro-sensors. Most of these micro-devices are mainly fabricated on a biochemical chip for performing procedures such as pre-sampling treatment, mixing, transfer, isolation and detection. When a micro-fluidic chip is used to carry out a biomedical inspection or analysis, the advantages over manual operation include fewer experimental errors, higher system stability, lower power consumption and sampling quantities, lesser manual labor and shorter testing period.

In general, the method of forming the micro-fluidic chip includes applying the semiconductor etching technique to etch out micro-channels in a glass or plastic substrate. The sample to be inspected is allowed to pass through the micro-channels and the necessary chemical reactions such as solution mixing and molecule separation are carried out sequentially. In other words, the entire biochemical lab function is established within the micro unit. Furthermore, because the inspection or analysis performed through the micro-fluidic chip often has to be carried out within a specific temperature range, a heating device is often required.

The simplest and most basic method of heating includes using an external heat source to heat up the entire system directly. However, the principal defect for this type of heating method is that a lot of power is wasted and areas not requiring the heating are also heated. Therefore, with the maturity of micro-electromechanical techniques, a micro-electromechanical heating element is formed so that a micro area can be directly heated. Because the heating takes place in a micro area, if the length, width and thickness of the resistive heating element are not properly designed, the difference in temperature within the heating area will be significant. Furthermore, whether the heating is carried out through the conventional method or by means of a micro-electromechanical heating element, the entire system is heated.

### SUMMARY OF THE INVENTION

Accordingly, the present invention provides a micro-scale heating module so that the temperature distribution of a working region of a micro-fluidic chip is more uniform and the probability of the difference in temperature affected by the fluid is minimized.

In one embodiment, the invention provides a micro-scale heating module for heating a micro-fluidic chip. The micro-fluidic chip typically includes an inlet, an outlet and a working region between the inlet and the outlet. The micro-scale heating module includes a preheating part and a heating part. The preheating part is correspondingly disposed on the inlet of the micro-fluidic chip. The heating part connects with the preheating part and surrounds the working region of the micro-fluidic chip in order to make the temperature distribution in the working region uniform.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A is a sketch of a micro-fluidic chip having a micro-scale heating module according to the first embodiment of the present invention.

FIG. 1B is a sketch showing the simulated distribution of temperature points when the module in FIG. 1A is heated up.

FIG. 2 is a sketch of a micro-fluidic chip having a micro-scale heating module according to the second embodiment of the present invention.

FIG. 3 is a sketch of a micro-fluidic chip having a micro-scale heating module according to the third embodiment of the present invention.

FIG. 4 is a sketch of a micro-fluidic chip having a micro-scale heating module according to the fourth embodiment of the present invention.

### DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

The micro-scale heating module in the present invention is used for heating a micro-fluidic chip. The main design concept is to divide the micro-scale heating module into a pre-heating part and a heating part. The pre-heating part is correspondingly disposed at an inlet of the foregoing micro-fluidic chip so that fluid is heated up to a higher temperature before entering a working region of the micro-fluidic chip. The heating part is disposed around the working region of the micro-fluidic chip so that any fluid within the working region is heated to a specific uniform temperature. In the following, a number of embodiments are described as examples. However, these embodiments should by no means limit the scope of the present invention.

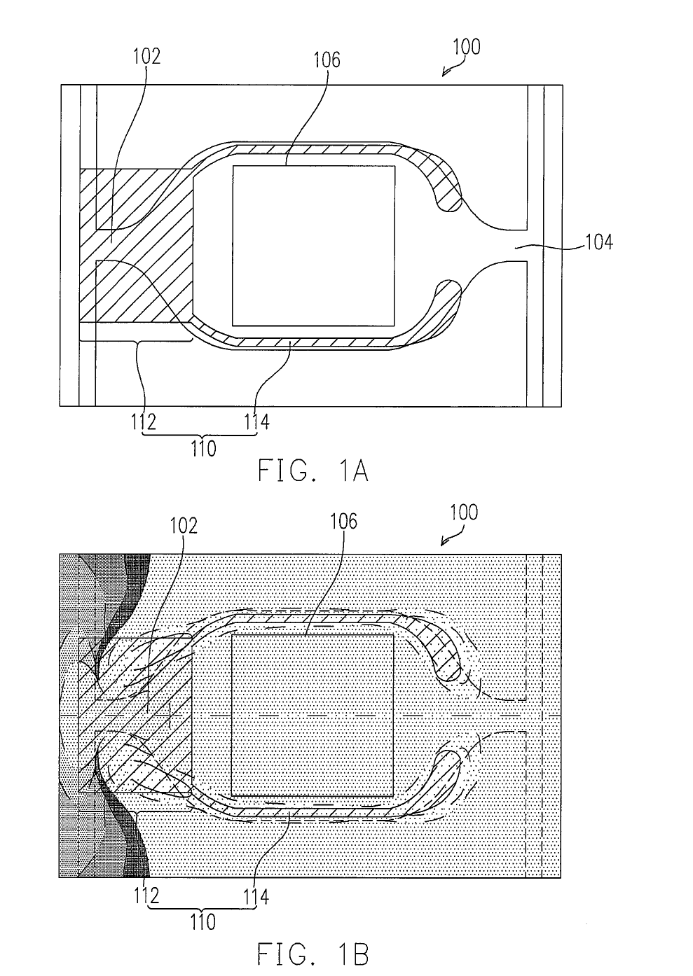

FIG. 1A is a sketch of a micro-fluidic chip having a micro-scale heating module according to a first embodiment of the present invention.

As shown in FIG. 1A, the present embodiment includes a micro-fluidic chip 100 having an inlet 102, an outlet 104 and a working region 106. The working region 106 is disposed between the inlet 102 and the outlet 104. In the present embodiment, the micro-scale heating module 110 comprises a pre-heating part 112 and a heating part 114. The pre-heating part 112 is disposed in a location, for example, at the inlet 102 of the micro-fluidic chip 100 and overlapping the inlet 102. The heating part 114 connects with the pre-heating part 112 and surrounds the working region 106 of the micro-fluidic chip 100 so that the foregoing working region 106 can have a uniform temperature distribution. The heating part 114 separates from the working region 106 by a distance. When the micro-scale heating module 110 of the present invention is installed inside an ordinary chip like the micro-fluidic chip 100, fluid can flow at a suitable speed into the chip so that the fluid within the working region 106 is maintained at a constant temperature.

In general, the efficiency of heat exchange is higher in a convective heat transfer mode than a conductive heat transfer mode. Therefore, the foregoing embodiment can be used to reduce the temperature gradient along the direction of flow of the fluid within the working region 106 through a suitable setting of the flow rate of the fluid and a suitable positioning of the pre-heating part before the working region 106. Furthermore, since the conventional heating source will lead to a significant temperature gradient, the main heating part 114 is shifted towards the outer edge of the working region 106 in order to minimize the temperature gradient within the working region 106 and maintain a uniform temperature distribution. Moreover, the embodiment of the present invention also matches the fluid flow direction such that no heating element is set up downstream of the working region 106 (near the outlet 104). Instead, the heat from the high temperature fluid is used to heat up this area so that some power is saved. In the following, the effect provided by the present invention is verified through a computer simulation.

FIG. 1B is a sketch showing the simulated distribution of temperature points when the module in FIG. 1A is heated up. The areas with a lower pattern density represent parts having a higher temperature while the areas with a higher pattern density represent parts having a lower temperature.

As shown in FIG. 1B, assume the fluid flowing into the inlet of the micro-fluidic chip 100 has been kept in an environment maintained at a low temperature. Therefore, the fluid at the inlet 102 has a low temperature. Because of the low temperature, the pre-heating part 112 of the micro-scale heating module 110 is maintained at a higher temperature so that the fluid is increased to a higher temperature before flowing into the working region 106. In other words, the fluid temperature in the pre-heating part 112 is higher than other parts (for example, the heating part 114 or the working region 106). Under these circumstances, the working region 106 of the micro-fluidic chip 100 is maintained at a relative uniform temperature. Furthermore, the diagram only shows the result of a simulation. If the design is further optimized through computation, a more uniform temperature distribution can be achieved.

In addition, the temperature equalizing function of the module is closely related to the material forming the micro-fluidic chip, the geometric dimension of the micro-fluidic chip, the specific gravity, the viscosity, the flow rate of the fluid as well as the shape of the micro-channels within the micro-fluidic chip. Furthermore, the material, the thickness, the length and the width of the heating element also affects the temperature distribution function. Therefore, the aforementioned factors can be used as control parameters for designing the module in the present invention.

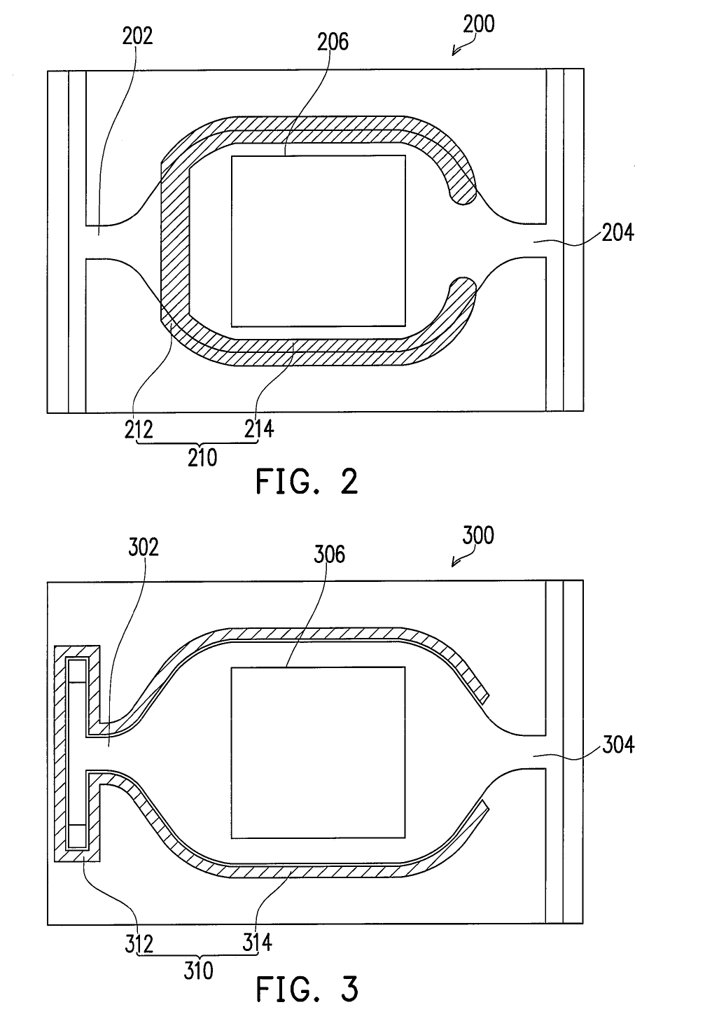

FIG. 2 is a sketch of a micro-fluidic chip having a micro-scale heating module according to a second embodiment of the present invention.

As shown in FIG. 2, the present embodiment has a micro-fluidic chip 200 identical to the first embodiment (including an inlet 202, an output 204 and a working region 206). However, the pre-heating part 212 of the micro-scale heating module 210 only partially overlaps the inlet 202 of the micro-fluidic chip 200, and the heating part 214 surrounding the working region 206 occupies a larger area than that in the first embodiment.

FIG. 3 is a sketch of a micro-fluidic chip having a micro-scale heating module according to the third embodiment of the present invention.

As shown in FIG. 3, the micro-fluidic chip 300 is almost identical to the one in the first embodiment (including an inlet 304 and a working region 306). The only difference lies in the shape of the inlet 302. The pre-heating part 212 of the micro-scale heating module 310 surrounds the inlet 302 of the micro-fluidic chip 300 and the heating part 214 surrounds the working region 306.

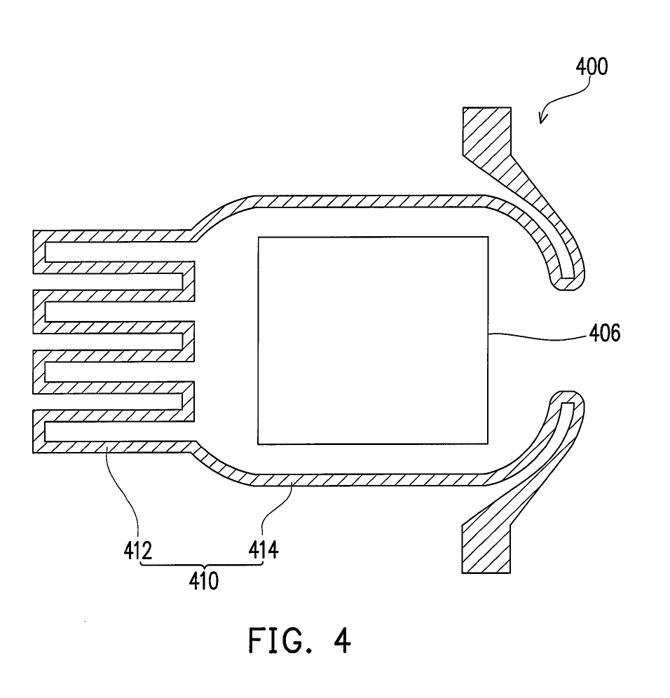

FIG. 4 is a sketch of a micro-fluidic chip having a micro-scale heating module according to the fourth embodiment of the present invention.

As shown in FIG. 4, the present embodiment uses the working region 406 of the micro-fluidic chip 400 only as a relative positional base for the micro-scale heating module 410. For example, the heating source of the micro-scale heating module 410 is typically a resistor heating element. Therefore, in the present embodiment, the pre-heating part 412 is formed using a line of heating element bending back and forth multiple times. Furthermore, when the flow rate of the fluid is increased, multiple electrode leads can be used to change the area size covered by the pre-heating part 412 and hence the heating rate. For example, when the flow rate of fluid inside the micro-fluidic chip 400 is large, the pre-heating part 412 is designed to cover a larger area. Conversely, when the flow rate of fluid inside the micro-fluidic chip 400 is small, the pre-heating part 312 is designed to cover a smaller area.

In summary, one major feature of the present invention is the installation of the specially shaped micro-scale heating module inside a micro-fluidic chip so that a stable and uniform temperature region is formed after the fluid flowing into the micro-fluidic chip. Furthermore, even when the flow rate of the fluid changes, a uniform temperature is maintained within the working region. Thus, the micro-scale heating module of the present invention has the advantages of a simple design, the capacity to work under a large range of flow rates and a relatively large working region. The micro-scale heating module is particularly useful in applications such as cell culture, cell-to-pharmaceutical test or biochemical test, just to name a few.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A micro-scale heating module for heating a micro-fluidic chip having an inlet, an outlet and a working region, wherein the working region is disposed between the inlet and the outlet, comprising:
a pre-heating part, disposed to correspond with the inlet of the micro-fluidic chip; and
a heating part connected to the pre-heating part and surrounding the working region of the micro-fluidic chip so that the working region has a uniform temperature distribution.

2. The micro-scale heating module of claim 1, wherein the pre-heating part overlaps the inlet of the micro-fluidic chip.

3. The micro-scale heating module of claim 1, wherein the pre-heating part surrounds the inlet of the micro-fluidic chip.

4. The micro-scale heating module of claim 1, wherein the heating part separates from the working region of the micro-fluidic chip by a distance.

5. The micro-scale heating module of claim 1, wherein, when the flow rate of fluid inside the micro-fluidic chip is large, the pre-heating part is designed with a larger area.

6. The micro-scale heating module of claim 1, wherein, when the flow rate of fluid inside the micro-fluidic chip is small, the pre-heating part is designed with a smaller area.
